(19) United States
(12) Patent Application Publication
(10) Pub. No.: US 2026/0118151 A1
(43) Pub. Date: Apr. 30, 2026

(54) DETECTION DEVICE FOR DETERMINING A RELEASE CRITERION FOR A FILL LEVEL DETERMINATION SYSTEM

(71) Applicant: Elixion Medical GmbH, Duesseldorf (DE)

(72) Inventors: Mario ROSER, Muenchen (DE); Ludwig FENDT, Muenchen (DE)

(73) Assignee: Elixion Medical GmbH, Duesseldorf (DE)

(21) Appl. No.: 19/421,553

(22) Filed: Dec. 16, 2025

Related U.S. Application Data
(63) Continuation of application No. PCT/EP2024/066652, filed on Jun. 14, 2024.

(30) Foreign Application Priority Data
Jun. 16, 2023 (DE) 10 2023 115 879.1

(51) Int. Cl. G01F 23/263; A61F 5/44

(52) U.S. Cl. CPC G01F 23/263; A61F 5/4405

(57) ABSTRACT

A fill level determination system and detection device for determining a release criterion for a fill level determination system is provided, which determines a fill level of a medical fluid in a container. A position detector determines a position of the fill level determination system. An acceleration detector determines an acceleration of the fill level determination system. Fill level measurement values of the fill level determination system are recorded as a plausibility measurement, if the determined position lies within a tolerance range around a reference position and if the acceleration lies within a tolerance range. The release criterion is granted on the basis of the plausibility measurement if a fill level measurement value of the fill level determination system does not show an excessive change from a previous fill level measurement value.

This nonprovisional application is a continuation of International Application No. PCT/EP2024/066652, which was filed on June 14, 2024, and which claims priority to German Patent Application No. 102023115 879.1, which was filed in Germany on June 16, 2023, and which are both herein incorporated by reference.

### BACKGROUND OF THE INVENTION

### Field of the Invention

The present invention relates to a fill level determination system for determining a release criterion of a medical fluid in a container. Furthermore, the present invention relates to a detection device, a container for a fill level determination system, and a method for determining a release criterion for a fill level determination system.

### Description of the Background Art

Containers for collecting medical fluids are generally known. These containers can be urine bags or urine measurement systems used to collect a patient's urine for the purpose of monitoring and caring for the patient. Furthermore, the containers can be used to collect the patient's fluids during drainage procedures, for example fluids that need to be removed from wounds after surgery. In addition, the containers can deliver medical fluids to a patient, for example as infusions (infusion bags). When using any of these types of containers that collect medical fluids, determining the change in the fill level in the container over the course of treatment is always part of monitoring and caring for the patient.

Patient monitoring and care can take place in hospitals, on regular wards, in intensive care units, in nursing homes, or in home care settings.

It is also known that a scale for displaying the fill level is provided on the containers. Devices for measuring the fill level are also known. These use various measuring methods, such as weighing or capacitive determination of the fill level.

US 2020/0209044 A1 discloses a printed circuit board device comprising: a first capacitive sensor configured to measure a first capacitance within a self-contained volume having known dimensions, wherein the first capacitance changes as a substance is fed into the self-contained volume; a second capacitive sensor having a plurality of activation points at a plurality of corresponding known heights within the self-contained volume, wherein the second capacitive sensor is configured to detect when the substance fed into the self-contained volume has reached the corresponding known heights, respectively; and wherein at least one of a fill level of the substance within the self-contained volume, a volume of the substance within the self-contained volume, or a flow rate of the substance into the self-contained volume is determined based on data from the first capacitive sensor and the second capacitive sensor.

### SUMMARY OF THE INVENTION

It is therefore an object of the present invention is to provide an improved system for determining the fill level of a medical fluid in a container.

When measuring a fill level, a position, acceleration, or compression of the container may change the fill level and thus also cause a measurement error in determining the fill level. Furthermore, some containers for collecting a medical fluid are flexible, for example bags. Since flexible bags cannot stand upright on their own, an accurate fill level measurement is only possible if they have first been placed in a defined vertical hanging position, usually at the patient's bed, using a holding system. However, when transporting the patient in their bed, the bag is often removed and placed on the patient's bed, resulting in the bag deforming freely and making a reliable fill level measurement impossible.

Strong acceleration can also lead to measurement errors. These can occur, for example, when the patient is already mobile again and for instance walks to the toilet with the container. The acceleration due to walking causes changes in the position of the container and wave formation in the container, making a reliable fill level measurement impossible.

In addition, compressing the container can also lead to a measurement error. Such errors can occur, for example, if the patient compresses the urine bag hanging on the side of the bed, e.g., by pulling a bedside table on casters closer to the bed, causing the urine bag to become wedged between the bed and the bedside table.

To solve the problem of these measurement errors, a detection device according to the invention is provided which only allows a fill level measurement, i.e., determines a release criterion, if the above measurement errors are not to be expected.

Such a detection device is advantageous for fill level measurements for many different containers in the above-mentioned areas of application. In order to be able to use this detection device with the various containers, a fill level determination system according to the invention is provided, which comprises the detection device and is modular, so that it is possible to determine a release criterion for a fill level measurement for many different types of containers.

According to a first aspect, the present invention provides a detection device for determining a release criterion for a fill level determination system.

According to a second aspect, the present invention provides a fill level determination system for determining a fill level criterion of a medical fluid in a container.

According to a third aspect, the present invention represents a container for a fill level determination system.

According to a fourth aspect, the present invention provides a method for determining a release criterion for a fill level determination system.

Further scope of applicability of the present invention will become apparent from the detailed description given hereinafter. However, it should be understood that the detailed description and specific examples, while indicating preferred embodiments of the invention, are given by way of illustration only, since various changes, combinations, and modifications within the spirit and scope of the invention will become apparent to those skilled in the art from this detailed description.

### BRIEF DESCRIPTION OF THE DRAWING

The present invention will become more fully understood from the detailed description given hereinbelow and the accompanying drawings which are given by way of illustration only, and thus, are not limitive of the present invention, and wherein:

FIG. 1 is a fill level determination system according to an example;

FIG. 2 is a control device for a fill level determination system according to an example;

FIG. 3a is a control device according to an example;

FIG. 3b is a control device according to an example;

FIG. 4a is a fill level measurement arrangement according to an example;

FIG. 4b is a fill level measurement arrangement according to an example;

FIG. 5a is a fill level measurement arrangement according to an example;

FIG. 5b is a fill level measurement arrangement according to an example;

FIG. 5c is a fill level measurement arrangement according to an example;

FIG. 5d is a fill level measurement arrangement according to an example;

FIG. 6a is a container according to an example;

FIG. 6b is a container according to an example;

FIG. 6c is a container according to an example;

FIG. 6d is a container according to an example;

FIG. 6e is a container according to an example;

FIG. 6f is a container according to an example;

FIG. 6g is a container according to an example;

FIG. 6h is a container according to an example;

FIG. 7 is a control device for a fill level measurement arrangement according to an example;

FIG. 8 is a control device, a fill level measurement arrangement and a container according to an example;

FIG. 9a is a control device, a fill level measurement arrangement and a container according to an example;

FIG. 9b shows a control device, a fill level measurement arrangement and a container according to an example;

FIG. 9c shows a cross-section through the pouch of the container with the fill level measurement arrangement and the rear clamping device according to the example shown in FIG. 9b;

FIG. 9d shows a cross-section through the pouch of the container with the fill level measurement arrangement according to an example;

FIG. 9e shows a control device, a fill level measurement arrangement and a container according to an example in a side view;

FIG. 9f shows a control device, a fill level measurement arrangement and a container according to an example in a side view;

FIG. 9g shows a cross-section through the pouch of the container with the fill level measurement arrangement according to an example;

FIG. 9h shows a cross-section through the pouch of the container with the fill level measurement arrangement according to the example shown in FIG. 9g;

FIG. 9i shows a rear side of the container according to an example;

FIG. 9j shows a rear side of the container according to an example;

FIG. 9k shows a control device, a fill level measurement arrangement and a container according to an example;

FIG. 9l shows a control device, a fill level measurement arrangement and a container according to an example;

FIG. 9m shows a control device, a fill level measurement arrangement and a container according to an example;

FIG. 10 is a control device, a fill level measurement arrangement and a container according to an example;

FIG. 11 is a control device and a fill level measurement arrangement according to an example;

FIG. 12 is a control device, a fill level measurement arrangement and a container according to an example;

FIG. 13 is a control device, a fill level measurement arrangement and a container according to an example;

FIG. 14 is a control device and a fill level measurement arrangement according to an example;

FIG. 15 is a control device, a fill level measurement arrangement and a container according to an example;

FIG. 16 is a flow diagram of an exemplary fill level measurement; and

FIG. 17 is an example of a multi-purpose computer.

### DETAILED DESCRIPTION

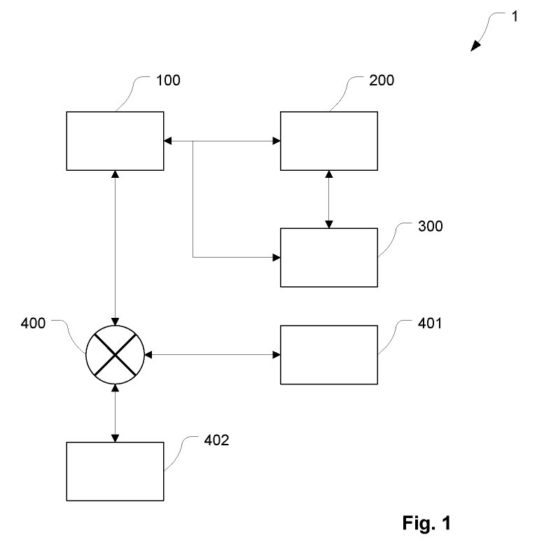

FIG. 1 illustrates an example in accordance with the present invention. Prior to a detailed description, general explanations of the examples are provided.

According to the invention, a detection device for determining a release criterion for a fill level determination system which determines a fill level of a medical fluid in a container, comprises: a position detector configured to determine a position of the fill level determination system, an acceleration detector configured to determine an acceleration of the fill level determination system, wherein fill level measurement values of the fill level determination system are recorded as a plausibility measurement, if the determined position lies within a tolerance range around a reference position and if the acceleration lies within a tolerance range, and the release criterion is granted on the basis of the plausibility measurement if a fill level measurement value of the fill level determination system does not show an excessive change from a previous fill level measurement value.

The position detector and the acceleration detector can be part of an inertial measurement unit (IMU). IMUs can have an accelerometer that measures acceleration along the three axes (including gravitational acceleration) and a gyroscope that measures the rate of angular change (in degrees per second) when rotating around the three axes.

Position detection can be performed using an accelerometer, for example, acceleration in a strictly vertical position along the z-axis corresponds to 1g, and acceleration on the x- and y-axes corresponds to 0g. The strictly vertical position is an example of a reference position. When tilted, the acceleration due to gravity is projected proportionally onto the x and y axes, allowing the position in space to be calculated. It is therefore easy to verify whether the container and IMU are in a vertical position (within the tolerance limits around the reference position). This applies as long as the relative orientation between container and IMU remains constant.

Tilted positions within the tolerance range are considered tolerable tilted positions, but if left uncorrected, they would cause a measurement error; however, they can be corrected (wherein the correction depends on the geometry of the container).

Acceleration detection can be performed using an accelerometer and a gyroscope, as both functional devices (accelerometer and gyroscope) must be used for measuring the six degrees of freedom of acceleration. Here, the accelerometer measures 3 translational degrees of freedom and the gyroscope measures 3 rotational degrees of freedom.

It is therefore possible to verify whether the container and the IMU are in a stationary position within the tolerance limits. However, if an excessive acceleration is detected, e. g., the patient is walking with the system, the release criterion is not granted and the measurement is rejected, as the same acceleration also acts on the liquid in the container and can induce wave movements, resulting in an inaccurate measurement.

The release criterion cannot be granted based on the measurement of the fill level in the container either, for example if an excessive change in the fill level is measured. Here, an excessive increase is, for example, a change in fill level that exceeds a predetermined threshold value. This is also time-dependent. Between two fill level values that are one hour apart a larger change is plausible than between two fill level values that are one minute apart. Thus, an excessive change may occur if a gradient of the fill level over time is exceeded (or not reached) between the fill level measurement value and the previous fill level measurement value. Alternatively, a different threshold value can be used for different time intervals between the fill level measurement value and the previous fill level measurement value.

If acceleration and position are within the tolerances and no excessive increase is detected, the release criterion is granted and the determined measurement values are classified as plausible. In this way, a fill level criterion can be determined from the plausible measurement values.

According to the invention, a fill level determination system for determining a fill level criterion of a medical fluid in a container comprises: the container, which is configured to hold a medical fluid; a fill level measurement arrangement, which is configured to record measurement values corresponding to a fill level criterion of the container, a detection device as described herein, and a control device, wherein the control device and the fill level measurement arrangement are configured to be connectable to each other and the fill level measurement arrangement and the container are configured to be connectable to each other, wherein the determination of the fill level criterion takes place in a state of connection between the control device and the fill level measurement arrangement and between the fill level measurement arrangement and the container.

The control device may have connectivity. This can be implemented using integrated wireless communication technology, such as Bluetooth (including BLE), wireless LAN (WLAN), RFID (including NFC), or mobile communications, to transmit measurement data. This may involve implementation of an online function, in which data is transmitted to a hospital information system (HIS) or patient data management system (PDMS), and an offline function, in which data is temporarily stored locally and then transferred the next time the connection or online function (push) is available.

The fill level determination system can record the fill level in the container (including emptying) and output the fill level, for example in millimeters; for this purpose, a display can be provided or the fill level can be transmitted to a server or end device via a communication unit. The fill level measurement can have an accuracy of +/- 5%, for example.

The fill level determination system integrates a position sensor and an acceleration sensor in the detection device in order to distinguish between valid and invalid (e.g., tilted position, movement, compression) measurement conditions. In this way, the release criterion can be determined.

The fill level determination system can integrate one or more temperature sensors that measure the ambient temperature.

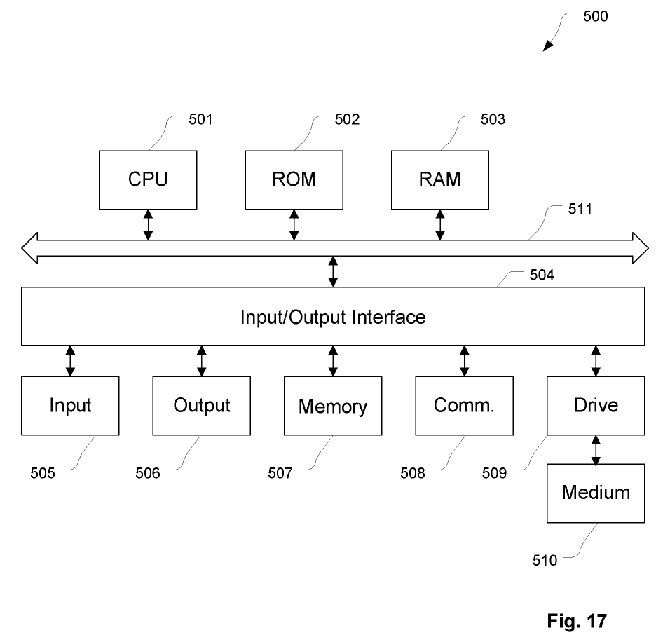

The fill level determination system can also provide computing power and memory (SRAM, flash, SD card) for local data processing. This may be provided, for example, when implementing the offline function. Furthermore, these can be used for the calculations in steps S12 to S17 (FIG. 17).

The fill level determination system may also comprise a battery, together with (wireless) charging and monitoring electronics to supply power to the system. This type of energy management enables operation on battery power. Battery operation can also be provided by plug-in charging or wireless charging via induction.

A pairing of at least two of the control device, the container, and the fill level measurement arrangement enables a compatibility check. In addition, pairing can be used to establish a coupling to a patient or an electronic patient record (on a server). For pairing, an RFID device may be provided, which is disposed in the control device, container, and fill level measurement arrangement, respectively.

In order to enable assessment of the fluid properties in poor lighting conditions, container lighting may be provided to illuminate the container and the fluid inside it. This lighting can be activated, for example, by detecting an approach or a gesture, for example for a specific time interval. Similarly, the display can be switched on from standby mode by detecting an approach or a gesture.

Connectivity allows end devices such as smartphones or smartwatches to be connected, for example via an app. In this way, the measurement values can be transmitted to the nursing staff on an end device and displayed with the corresponding generated visualizations. The visualizations can be generated on a server or the end device, for example, and can comprise a fill level history over time. This server can also comprise the HIS or PDMS. Furthermore, the nursing staff can be notified of a need for action via notifications. An example of a need for action is a full container, wherein additional patient data can be provided, which also comprises a specific room in the hospital or care facility.

The approximate location of the fill level determination system can also be displayed to the nursing staff, such as a specific device in a specific building on a specific floor in a specific room.

Connectivity via RFID enables an RFID device on the smartphone to be activated and read an RFID tag on the container, thereby determining whether the container is in use or unused, which patient the container is assigned to, or how long the container has been in use.

Connectivity via RFID enables communication with the control device by activating an RFID device on the smartphone, e.g., for setup processes in which a connection to a wireless network is established, patient data is transferred, or a connection to a patient record is created.

To ensure modularity between the control device and the fill level measurement arrangement, a holding mechanism for an exchangeable fill level measurement arrangement may be implemented. The holding device also establishes an electrical connection for data transmission, for example via a standardized interface. The holding mechanism thus serves as a communicating frame.

The fill level determination system can communicate with a server that receives data from the various devices, wherein each of the devices comprises a container, a fill level measurement arrangement, a control device, and a detection device. The server can be operated in the local network or the cloud, and data can be stored, visualized and analyzed, as well as forwarded to hospital information systems or smartphones/smartwatches and stored there. The server may be comprised in the fill level determination system.

With the end devices, RFID technology makes it possible to read and write RFID tags on the container by activating the smartphone's RFID device.

To enable battery operation, a charging infrastructure may also be comprised in the fill level determination system. This may comprise a wall shelf, a rack, etc. for storage with integrated charging capability. The transfer of electrical energy during charging can be achieved through inductive charging via pads, inductive charging via “puck” — which is moved into the correct position by magnets — or a contact plug with magnetic positioning.

There are examples wherein the control device and the fill level measurement arrangement are configured to be connectable to each other via a standardized interface.

There are examples wherein the container comprises a holder and the fill level measurement arrangement and the container are configured to be connectable to each other via the holder.

There are examples wherein the holder on the container comprises a flexible pouch that is designed to accommodate the fill level measurement arrangement based on a sword-sheath principle.

The holder for the fill level measurement arrangement on the container can be attached to the front or back of the bag, for example, or more precisely to any surface of the container that is parallel to the vertical. The reference position for determining the location is derived from the installation.

The holder can be a flexible, thin-walled, foil pouch cut as close as possible, e.g., by welding or gluing flexible foil to the wall.

There are examples wherein the fill level measurement arrangement comprises a rear clamping device that generates a clamping force in the flexible pouch.

The clamping force allows the foil of the pouch to be pulled taut, particularly on the inside between the fill level measurement arrangement and the fluid in the container. In this way, air bubbles that could distort the measurement are avoided. Due to its dielectric constant, air is suitable as an insulator for capacitive measurements, which means that air bubbles distort the measurement. The clamping device can generate a clamping force both orthogonally and parallel to the insertion direction of the pouch.

There are examples wherein the container comprises a counter bearing that is configured to fasten the rear clamping device in the pouch.

There are examples wherein the rear clamping device is designed with a latching element and the latching element (207) latches into the counter bearing (309) so that the clamping force is generated.

Due to the latching element, a clamping force can be generated along the insertion direction of the pouch.

There are examples wherein the rear clamping device is designed with a semitubular-like profile.

Due to the semitubular-like profile, an air cushion is created along the entire rear side of the fill level measurement arrangement, shielding the fill level measurement arrangement from the exterior outside the container. Thus, the insulating effect of the air can be utilized here.

There are examples wherein the rear clamping device is inflatable. This also allows for an air cushion along the entire rear side of the fill level measurement arrangement.

There are examples wherein the rear clamping device comprises a spring device. Here, the spring force can generate the clamping force. In addition, a holding mechanism may be comprised that holds the rear clamping device in a compressed position of the spring device. For example, for inserting or removing the rear clamping device.

There are examples wherein the latching element is designed as a lever that latches into the counter bearing, thereby strutting the rear clamping device apart from the fill level measurement arrangement so that the clamping force is generated.

There are examples wherein the rear clamping device is designed to be bendable and can be latched into the counter bearing in a bent state.

There are examples wherein the holder on the container comprises a stiffened pouch that is designed to accommodate the fill level measurement arrangement based on a sword-sheath principle.

The stiffened pouch can be a plastic “sword sheath,” e.g., a stiffened plastic part welded or glued to the wall.

There are examples wherein the stiffened pouch comprises a stiffening on a wall towards the inside of the container.

There are examples wherein the stiffening is a frame.

There are examples wherein the fill level measurement arrangement and the container are configured to be connectable to each other via adhesion.

Fastening via adhesion can be carried out using double-sided adhesive tape, for example, which can be attached in advance to the fill level measurement arrangement, which is disposed of with the container after use, for example.

There are examples wherein the fill level measurement arrangement is printed on the container, whereby the fill level measurement arrangement and the container are configured to be connectable to each other.

All types of connection between the fill level measurement arrangement and the container must bring the sensor reliably into contact with the container wall, as air is a good dielectric insulator and varying air cushions between the sensor and the container wall would therefore falsify at least a capacitive measurement of the container fill level. A mechanism, for example a spring leaf, can be integrated into the rear inner wall of the pouch or sword sheath, which presses the sensor firmly against the container wall and thus ensures a repeatable positioning.

There are examples wherein the container comprises a urine bag.

There are examples wherein the container comprises a urine measurement system.

There are examples wherein the container comprises an infusion bag.

There are examples wherein the container comprises a thoracic drainage.

There are examples wherein the container comprises a ventricular drainage.

There are examples wherein the container comprises a surgical drainage.

Other containers are used, for example, for drainages in bag form, drainages in bottle form, infusion bottles and gastric tubes.

In general, the container can comprise a drain valve, e.g. a pinch valve, which can be actuated by an electromechanical device, for example on the fill level measurement arrangement.

There are examples wherein the container comprises an RFID device.

The RFID device can enable a coupling of the container with patients, the fill level measurement arrangement and the control device. This coupling also allows containers to be excluded, for example for compatibility reasons.

The RFID device can enable a presence detection of the container by the control device or the fill level measurement arrangement. Furthermore, identification of the container can be enabled, as well as writing of patient data to the RFID device of the container, matching of the compatibility of the container and the fill level measurement arrangement, matching of the container and the associated patient, exclusion of mix-ups between patients, exclusion of reuse of used containers, exclusion of the use of unauthorized consumables (non-original, expired, recall, etc.).

The coupling of patient and container can be triggered automatically when the RFID device of the container is detected. In addition, by coupling the control device with the RFID device of the container, the patient information (e.g. name, date of birth) can be automatically transferred and written to the RFID device of the container.

There are examples wherein the control device is configured to read the RFID device of the container via a further RFID device and to determine whether the container is compatible with the fill level measurement arrangement and the control device and has not yet been used, so that if the container is compatible and has not yet been used, it is written to the RFID device of the container via the RFID device that the container has (now) been used.

There are examples wherein by writing to the RFID device via the further RFID device of the container, patient-related data is also written to the RFID device of the container.

There are examples wherein the control device comprises the further RFID device.

The control device thus integrates an RFID device to identify containers and enable bidirectional communication with end devices such as smartphones.

There are examples wherein the container comprises an outlet valve and the fill level measurement arrangement comprises an actuator that is configured to actuate the outlet valve. The actuator can also be disposed separately from the fill level measurement arrangement and can be connected to the control device to communicate with it in order to be activated by it.

The actuator can be activated by the control device or an electronic component of the fill level measurement device, for example when the fill level in the container reaches a predetermined value, such as a maximum. Thus, an (electromagnetic) valve for automatic container emptying can be activated.

There are examples wherein the control device comprises a diagnostic device that is configured to analyze the fluid spectrometrically.

The spectrometric analysis can be provided, for example, by a mini spectrometer and a broadband radiation source. This is explained in more detail in WO2023/062224, for example.

There are examples wherein the control device comprises a display that is configured to display information on the determined fill level criterion.

The display can implement a human-machine interface (HMI) formed of a touchscreen and status LED and can be provided for the visualization of information and local operation.

A brightness sensor can be integrated to adjust the brightness of the display to the ambient brightness.

The status LED can signal a status of the fill level determination system or the patient – by evaluating the fill level measurement – if the display is in standby mode, for example. For this purpose, the status LED can use different color codes (green, yellow, red) or flashing codes.

The display can be designed as a touchscreen that can be operated with gloves.

There are examples wherein the control device comprises a presence detector that is configured to detect an approach of a person or a gesture of a person, wherein the display is switched on from a standby mode due to the approach or the gesture.

In this case, the person can be a user, e.g. the nursing staff. A gesture can be, for example, swiping with the hand above the display or swiping with the foot under the fill level determination system (if this is attached to the side of the patient’s bed).

There are examples wherein the control device comprises an energy management device that is configured to supply the control device with energy.

There are examples wherein the fill level measurement arrangement comprises electrodes that are configured to capacitively determine fill level measurement values via which the fill level criterion of the container can be determined.

Here, the measurement values of the electrodes can be corrected by environmental reference electrodes, fluid reference electrodes and temperature sensors, which record both the environmental and fluid temperature.

A standardized interface, e.g. I2C, SPI, UART or CAN bus, can be provided for connecting the fill level measurement arrangement to the control device. An additional electronic component can also be provided on the fill level measurement arrangement, which is formed to activate the sensors and an actuator. Furthermore, a fastening mechanism can be provided for interchangeable connection to the control device, resulting in a mechanical connection, while the interface provides the communication connection. This fastening mechanism is a plug-in connection, for example, and enables quick assembly of the (modular) fill level determination system.

The dimensioning of the fill level measurement arrangement can be adapted to different container sizes via different examples. The electrode design, as described herein, or the carrier material can also vary depending on the application. The fill level measurement arrangement can also be provided with or without an actuator that opens and closes a drain valve. There may be variants in which the fill level measurement arrangement, or at least the sensors such as the electrodes, are designed as a reusable part or disposable part, wherein the disposable part can be disposed of with the container after it has been glued on and used.

The fill level measurement arrangement may include one or more temperature sensors to measure the temperature of the liquid through the bag wall.

There are examples wherein the fill level measurement arrangement comprises a printed circuit board and the electrodes are formed on the printed circuit board.

There are examples wherein the printed circuit board is designed to be dimensionally stable.

There are examples wherein the printed circuit board is designed to be flexible.

The carrier material of the fill level measurement arrangement can be a rigid PCB, a flex PCB (enables adaptation to flexible or round container geometries) or printed electronics (also printed directly on the container; enables cost-effective disposable sensors).

There are examples wherein the fill level measurement arrangement comprises an electronic device that is configured to perform the determination of the measurement values using the electrodes.

In a capacitive measurement, for example, the sensor system of the fill level measurement arrangement comprises continuous electrodes (Texas Instruments, Ti FDC1004) that continue vertically along the container in the reference position. In addition, environmental reference electrodes and liquid reference electrodes can be comprised. The electrodes can also be designed to extend at an angle to the vertical direction.

Furthermore, instead of continuous electrodes, segmented electrodes with variable stages (Infineon PSoC Cap Sense) can be provided.

The electrodes can also be provided as parallel electrodes for measuring several columns of liquid next to each other. Alternatively and in combination, an alternative method of fill level measurement can be provided. For example, the fill level can be determined via inductive detection of a magnetic float in a riser tube or a vertical line of detectors (segmented presence detectors, e.g. optical detectors (diode array, CCD line) or an array of temperature sensors) or by measuring the weight of the bag. Other alternative methods can be standard procedures with e.g. a pressure sensor, a float, an ultrasonic sensor, a time-of-flight sensor or a laser.

An actuator can be comprised that is configured to perform a drain (drain valve) for emptying a container in the urine measurement system design via electromechanical activation. For this purpose, the actuator opens a valve of the container, for example a pinch valve, which the actuator can pinch in the closed state and does not pinch in the open state. The actuator can also comprise a pump that pumps a defined volume of fluid per cycle, for example.

There are examples wherein the control device is configured to perform a correction of the fill level criterion on the basis of the determined position of the position detector of the detection device when the release criterion is granted.

The control device is thus designed to correct position-dependent measurement errors when the container is suspended at an angle.

In accordance with the invention is a container for a fill level determination system according to the examples described herein.

In accordance with the invention is a method for determining a release criterion for a fill level determination system which determines a fill level of a medical fluid in a container, comprising the steps: Determining a position of the fill level determination system, determining an acceleration of the fill level determination system, recording fill level measurement values of the fill level determination system as a plausibility measurement if the determined position lies within a tolerance range around a reference position and if the acceleration lies within a tolerance range, and granting the release criterion on the basis of the plausibility measurement if a fill level measurement value of the fill level determination system does not show an excessive change from a previous fill level measurement value.

Returning to FIG. 1, this illustrates a fill level determination system according to an example.

The fill level determination system 1 comprises a control device 100, a fill level measurement arrangement 200 and a container 300. Furthermore, the fill level determination system 1 may comprise a network 400, a server 401 and an end device 402. The fill level determination system 1 has a modular design so that different control devices 100, fill level measurement arrangements 200 and containers 300 can be connected to each other or can be interchanged with each other.

The control device 100 is connected to the fill level measurement arrangement 200 and receives data indicative of a fill level in the container 300. These can be, for example, measurement values from the fill level measurement arrangement 200 that are read directly by the sensor system, a measured fill level or a measured fill level corrected for position. Furthermore, the control device 100 can receive information from the fill level measurement arrangement 200 that identifies the design of the fill level measurement arrangement 200.

The control device 100 and, alternatively or additionally, the fill level measurement arrangement 200 are connected to the container 300. Here, information on the design of the container 300 and whether the container has already been used is transmitted. In addition, the control device 100 and the fill level measurement arrangement 200 can transmit patient-specific information to the container 300 and write it to a memory that may be comprised on the container 300 (via RFID).

The control device 100 can communicate with the server 401 and the end device 402 via the network 400. The server 401 and the end device 402 can also communicate with each other via the network.

Thus, the control device 100 may transmit fill level measurement values (the fill level criterion) and information identifying the control device 100 or the patient, such as an identification number (ID) of the device or patient, to the server 401 and the server 401 may store these fill level measurement values in the corresponding patient record. Furthermore, the control device 100 can transmit fill level measurement values (the fill level criterion) and information identifying the control device 100 or the patient (ID) to the end device 402, wherein the end device 402 is assigned to the nursing staff in charge. The nursing staff in charge can be determined via the patient record (and a shift plan).

Both the server 401 and the end device 402 can be configured to display the fill level measurement values graphically. Either the control device 100, the server 401 or the end device 402 can determine a need for action, which can be communicated to the nursing staff in charge via the end device 402. A need for action can be, for example, an (almost) full container 300.

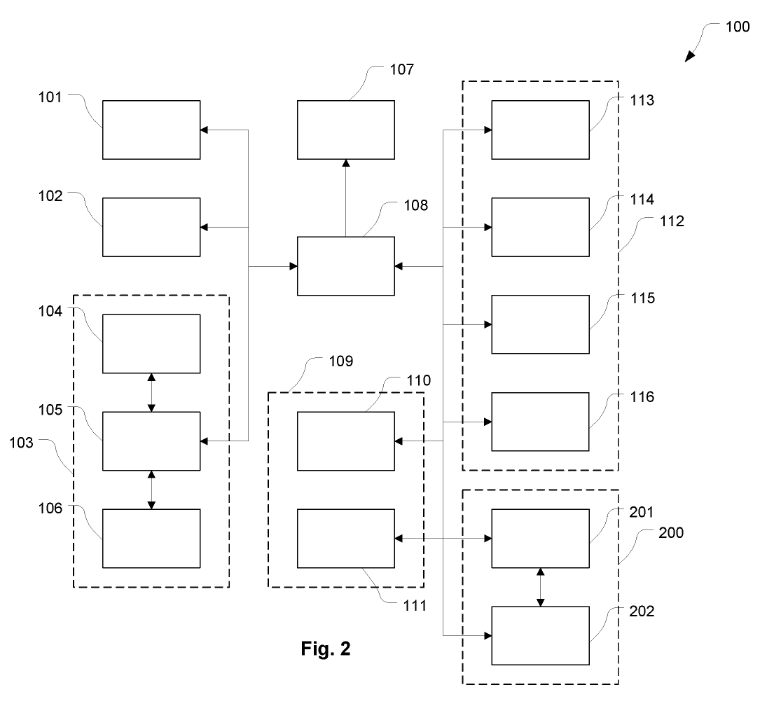

FIG. 2 shows a control device for a fill level determination system according to an example.

The control device 100 comprises a microcontroller 108, a display 107, a communication unit 109, a status LED 101, a container lighting 102, an energy management arrangement 103 and a sensor arrangement 112. Furthermore, the exchangeable fill level measurement arrangement 200 is also shown.

The microcontroller 108 is designed to perform the control functions of the control device 100. Furthermore, the microcontroller 108 can display information on the display 107. This information can include fill level measurement values, patient data or status information of the control device, such as the charge status. The microcontroller 108 can also have a memory on which data can be stored locally. The memory may also be comprised in the control device 100.

The display 107 may display the information and may also comprise a touch unit as an input device of the control device 100, with which a user (such as a nursing staff) may operate the control device 100.

A status of the patient, which is determined on the basis of the fill level measurement values, can also be indicated by the status LED 101. Furthermore, the status LED can also indicate a status of the control device 100 or the fill level determination system 1. Such a status can be, for example, that the position or the acceleration are outside the tolerance range or that an excessive change in the fill level has been recorded so that no fill level measurement is carried out (no release criterion). Furthermore, a missing connection to a network, a full container or a low charge status may also be indicated.

The container lighting 102 can be switched on in order to check the fluid in the container in poor lighting conditions (dark room), for example based on the color.

The energy management arrangement 103 comprises a charging device 104, a charging controller 105 and a battery 106. The charging controller 105 controls the energy input by the charging device 104 and the energy storage and output by the battery 106 in order to supply the control device 100 (and the fill level measurement arrangement 200) with energy.

The charging device 104 may be an inductive charging device that contactlessly receives energy from outside the control device 100. The charging device 104 can also receive energy via a plug.

The communication unit 109 comprises communication devices according to common standards, such as for WLAN 110 or mobile communications 111. A communication device for (low energy) Bluetooth can also be provided. This allows the control device 100 to communicate with devices such as servers and end devices directly or via a network.

The sensor arrangement 112 comprises an inertial measurement unit (IMU) 113, an NFC reader 114, a presence detector 115 and a diagnostic device 116.

The inertial measurement unit 113 measures the acceleration and position of the control device 100 and the associated fill level measurement arrangement 200 as well as the container (300 in FIG. 1). Thus, the inertial measurement unit 113 determines the acceleration and position for determining the release criterion of the fill level measurement by the fill level measurement arrangement 200.

The NFC reader 114 is an RFID device that can communicate with an RFID device in the container (300 in FIG. 1) or an end device (402 in FIG. 1). During communication, for example, information for identifying the control device 100, the fill level measurement arrangement, the container and the end device can be transmitted, for example to check compatibility between them. Patient data and a usage status of the container can also be transmitted during communication.

The presence detector 115 detects a presence or a gesture of a user (a nursing staff member). Based on the detection, the display 107 can then be switched on from a standby mode or the container lighting 102 can be switched on. The presence detector 115 can comprise a motion detector or a camera (infrared).

The diagnostic device 116 comprises a broadband radiation source and an optical detector, such as a spectrometer for spectrometrically analyzing the medical fluid flowing through a hose from or into the container 300.

Further, in the sensor arrangement 112, a brightness detector may also be disposed, which is configured to detect the brightness in the environment of the control device 100. Based on the brightness, the brightness of the display 107 can then be adjusted.

The standby mode of the display can be initiated based on a past time interval and, for example, the detected brightness.

The fill level measurement arrangement 200 comprises an electronic unit (electronic component) 201 with electrodes for fill level measurement and an actuator 202, which may be disposed in some embodiments. The actuator 202 operates an outlet valve on the container (300 in FIG. 1).

The actuator 202 can be activated by the microcontroller 108 or the electronic unit 201, for example when the measured fill level has reached a certain level and no inflow is currently detected.

The sensor arrangement 112 or parts of the sensor arrangement 112 can also be disposed on the fill level measurement arrangement 200, wherein evaluation steps in determining the release criterion can also be performed by the electronic unit 201.

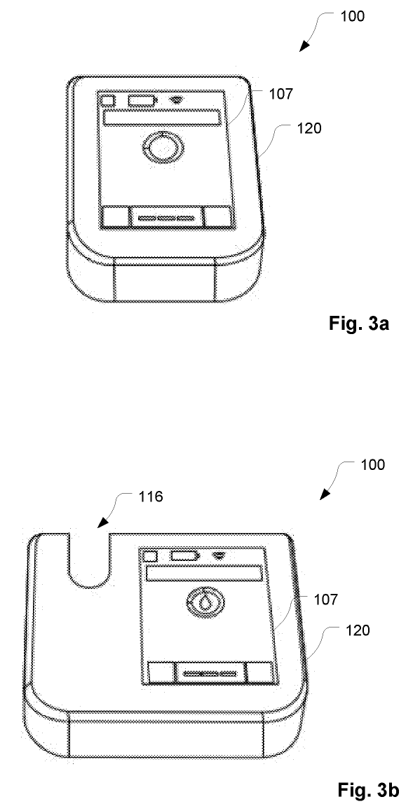

FIG. 3a shows a control device according to an example.

The control device 100 comprises a display 107 at the top of a housing 120, which comprises integrated circuits and arrangements that implement the functional blocks shown in FIG. 2.

FIG. 3b shows a control device according to an example.

The control device 100 comprises a display 107 at the top of a housing 120, which comprises integrated circuits and arrangements that implement the functional blocks shown in FIG. 2.

Furthermore, the control device 100 comprises the diagnostic device 116 in a housing section that can accommodate a hose leading into the container (300 in FIG. 1).

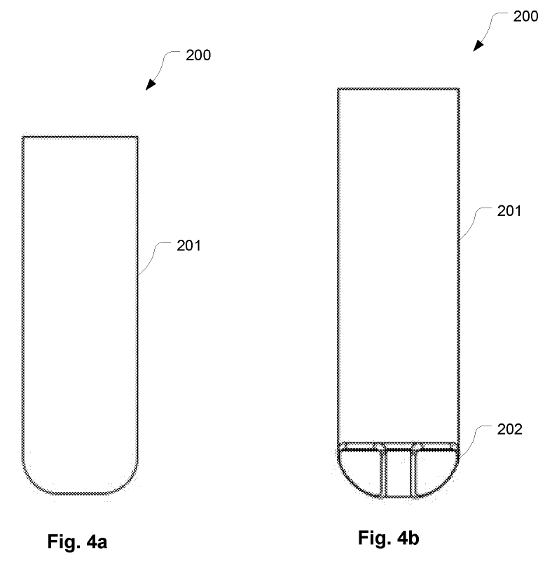

FIG. 4a shows a fill level measurement arrangement according to an example.

The fill level measurement arrangement 200 is designed as a test rod and comprises, in the test rod, a section that accommodates the electronic unit 201 with electrodes for the fill level measurement of the fill level in the container.

FIG. 4b shows a fill level measurement arrangement according to an example.

The fill level measurement arrangement 200 is designed as a test rod and comprises, in the test rod, a section that accommodates the electronic unit 201 with electrodes for the fill level measurement of the fill level in the container, as well as a second section that can function as a holder and accommodates the actuator 202.

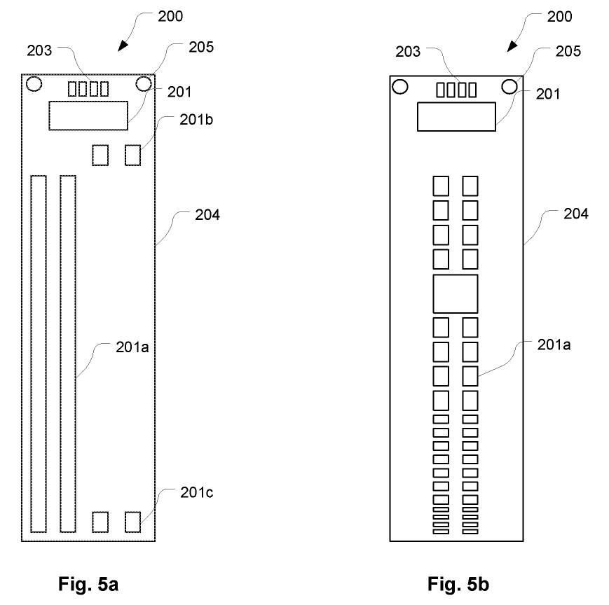

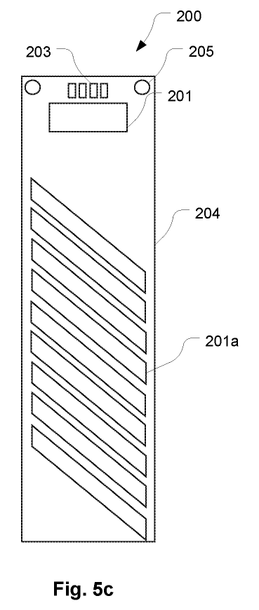

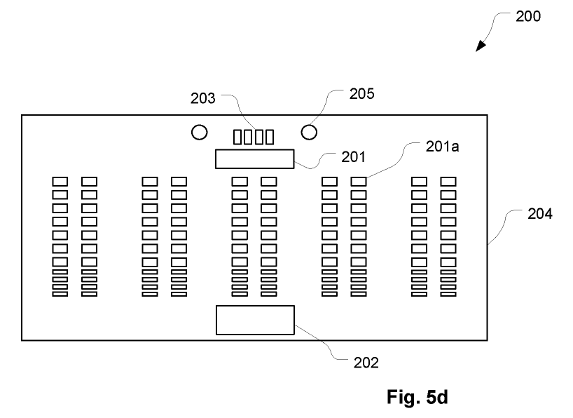

FIG. 5a shows a fill level measurement arrangement according to an example.

The fill level measurement arrangement 200 comprises an electronic unit 201, measuring electrodes 201a, ambient reference electrodes 201b, fluid reference electrodes 201c and a standardized interface 203 on a carrier material 204 and a fastening mechanism 205.

The carrier material 205 can be a flexible, rigid or printed printed circuit board. In the carrier material 205, the electrical connections of the components disposed on the carrier material 205 are designed.

The electronic unit 201 determines a capacitance between the measuring electrodes 201a, the ambient reference electrodes 201b and the fluid reference electrodes 201c respectively. The capacitance between the measuring electrodes 201a is corrected by the values measured by the reference electrodes.

The corrected capacitance values or a fill level determined therefrom by the electronic unit 201 can be output by the electronic unit 201 to the control device (100 in FIGS. 1 and 2) via the standardized interface 203.

The fill level measurement arrangement 200 is furthermore designed to be connectable to the control device by the fastening mechanism 205. The fastening mechanism 205 comprises, for example, pins that can be inserted into the control device.

FIG. 5b shows a fill level measurement arrangement according to an example.

The fill level measurement arrangement 200 comprises an electronic unit 201, segmented measuring electrodes 201a and a standardized interface 203 on a carrier material 204 and a fastening mechanism 205.

The carrier material 205 can be a flexible, rigid or printed printed circuit board. In the carrier material 205, the electrical connections of the components disposed on the carrier material 205 are designed.

The electronic unit 201 determines a capacitance between the segmented measuring electrodes 201a. The fill level can be determined by comparing the capacitances between the individual segments, which are disposed at the same height.

The fill level can be output by the electronic unit 201 to the control device (100 in FIGS. 1 and 2) via the standardized interface 203.

The fill level measurement arrangement 200 is furthermore designed to be connectable to the control device by the fastening mechanism 205. The fastening mechanism 205 comprises, for example, pins that can be inserted into the control device.

The reference electrodes shown in FIG. 5a are not shown but may also be included.

FIG. 5c shows a fill level measurement arrangement according to an example.

The fill level measurement arrangement 200 comprises an electronic unit 201, segmented measuring electrodes 201a and a standardized interface 203 on a carrier material 204 and a fastening mechanism 205.

The carrier material 205 can be a flexible, rigid or printed printed circuit board. In the carrier material 205, the electrical connections of the components disposed on the carrier material 205 are designed.

The electronic unit 201 determines a capacitance between the segmented measuring electrodes 201a. The fill level can be determined by comparing the capacitances between the individual segments.

The fill level can be output by the electronic unit 201 to the control device (100 in FIGS. 1 and 2) via the standardized interface 203.

The fill level measurement arrangement 200 is furthermore designed to be connectable to the control device by the fastening mechanism 205. The fastening mechanism 205 comprises, for example, pins that can be inserted into the control device.

The reference electrodes shown in FIG. 5a are not shown but may also be included.

FIG. 5d shows a fill level measurement arrangement according to an example.

The fill level measurement arrangement 200 comprises an electronic unit 201, segmented measuring electrodes 201a and a standardized interface 203 on a carrier material 204 and a fastening mechanism 205. The actuator 202 is also comprised.

The carrier material 205 can be a flexible, rigid or printed printed circuit board. In the carrier material 205, the electrical connections of the components disposed on the carrier material 205 are designed.

The electronic unit 201 determines a capacitance between the segmented measuring electrodes 201a. The fill level can be determined by comparing the capacitances between the individual segments, which are disposed at the same height. There are several columns of measuring electrodes 201a designed which can determine the fill level in several columns in a corresponding container (300 in FIG. 1).

The fill level can be output by the electronic unit 201 to the control device (100 in FIGS. 1 and 2) via the standardized interface 203.

The fill level measurement arrangement 200 is furthermore designed to be connectable to the control device by the fastening mechanism 205. The fastening mechanism 205 comprises, for example, pins that can be inserted into the control device.

The actuator 202 can be activated by the electronic unit 201 or the control device (100 in FIG. 2) to operate a drain valve of the container.

The reference electrodes shown in FIG. 5a are not shown but may also be included.

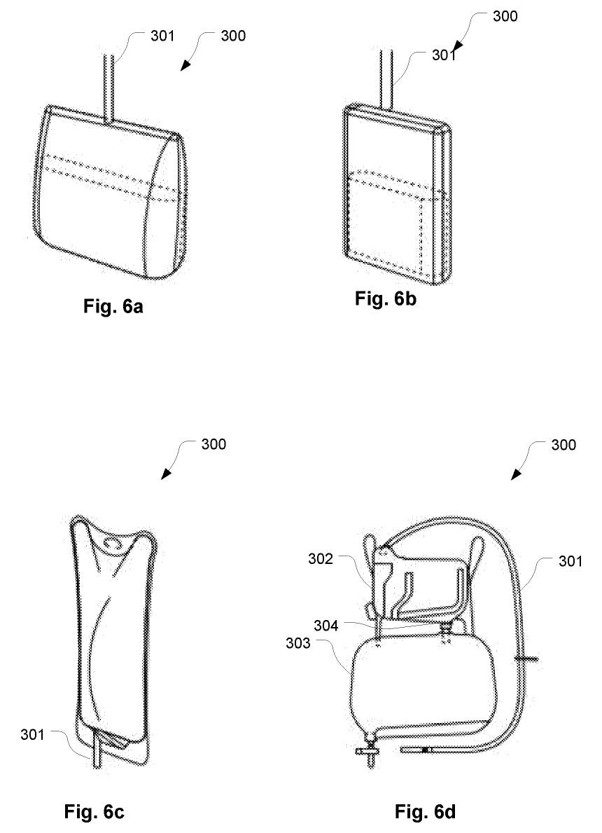

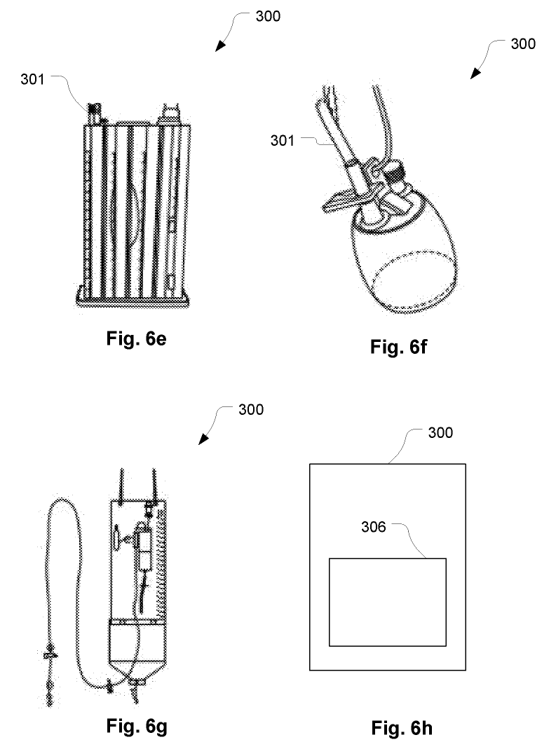

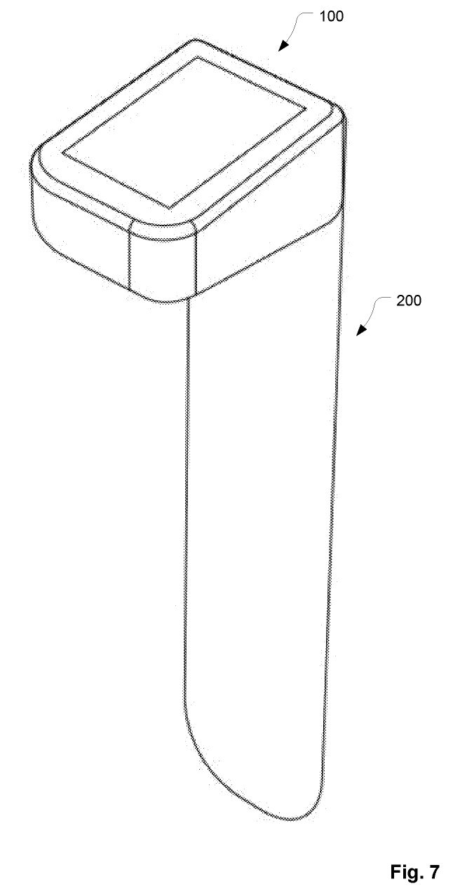

FIG. 6a shows a container according to an example.

The container 300 is designed with a hose 301 as an inflow. In the form shown, the container is a (urine) bag and is used to collect urine from a patient in a normal ward of a hospital or care facility.

FIG. 6b shows a container according to an example.

The container 300 is designed with a hose 301 as an inflow. In the form shown, the container is a drain and is used to collect fluids from a patient's wound, for example after an operation on the patient.

FIG. 6c shows a container according to an example.

The container 300 is designed with a hose 301 as a outlet. In the form shown, the container is an infusion bag and is used for storing and planned delivery of medication, nutrients and minerals into the patient's bloodstream.

FIG. 6d shows a container according to an example.

The container 300 is designed with a hose 301 as an inflow, an upper container 302, a lower container 303 and a transition 304. The upper container is used to measure the fill level, wherein the upper container has several columns in which liquid columns can accumulate, each of which can overflow into the next column. Fill level measurement can then be carried out with the several columns of electrodes of the fill level measurement arrangement according to FIG. 5d. Transition 304 comprises a valve that is operated by the actuator (202 in FIG. 5d). This valve can be a pinch valve, which only requires a flexible hose on the container 300. The actuator pinches the hose to close the valve. In the form shown, the container is a urine measurement system and is used to collect urine from a patient in a hospital intensive care unit.

FIG. 6e shows a container according to an example.

The container 300 is designed with a hose 301 as an inflow. In the form shown, the container is a thoracic drainage and is used to collect fluids from a patient's wound, for example after an operation on the patient. The container has several columns in which liquid columns can accumulate, each of which can overflow into the next column. Fill level measurement can then be carried out with the several columns of electrodes of the fill level measurement arrangement according to FIG. 5d.

FIG. 6f shows a container according to an example.

The container 300 is designed with a hose 301 as an inflow. In the form shown, the container is a surgical drainage in the shape of a bottle and is used to collect fluids from a patient's wound, for example after an operation on the patient.

FIG. 6g shows a container according to an example.

The container 300 is designed with a hose 301 as an inflow. In the form shown, the container is a ventricular drainage and is used to collect a patient's cerebrospinal fluid, for example after an operation on the patient.

FIG. 6h schematically shows a container according to an example.

The container 300 additionally comprises an RFID device 306, which may be applied (glued) to a surface of the container 300. The RFID device 306 may be an NFC tag and may store patient data and a usage status (whether the container 300 is already used). Furthermore, the RFID device 306 can also transmit an ID, wherein the usage status for this ID is stored, for example, on a server (401 in FIG. 1), control device (100 in FIG. 1) or end device (402 in FIG. 1).

The RFID device 306 may be applied to any of the containers 300 of FIGS. 5a to 5g.

FIG. 7 shows a control device and a fill level measurement arrangement according to an example.

The fill level measurement arrangement 200 is connected to the control device 100 at the rear. This connection is designed, for example, using a fastening mechanism and a standardized interface. The fill level measurement arrangement 200 shown is designed as in FIG. 4a and the control device 100 shown is designed as in FIG. 3a.

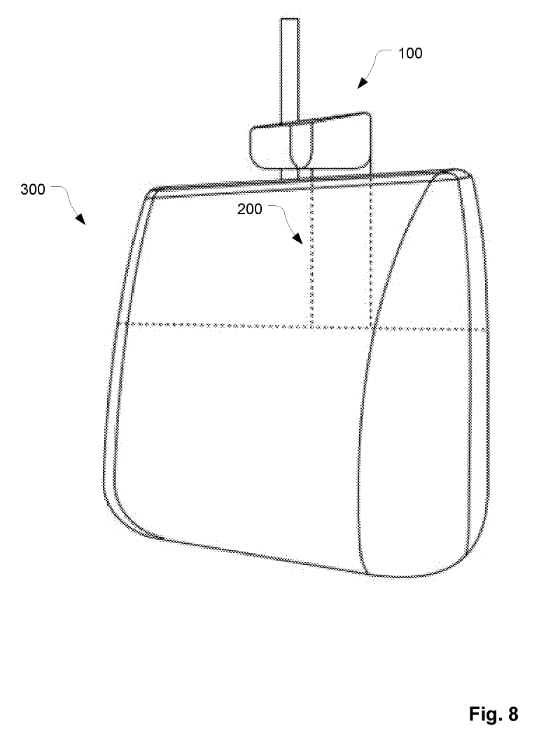

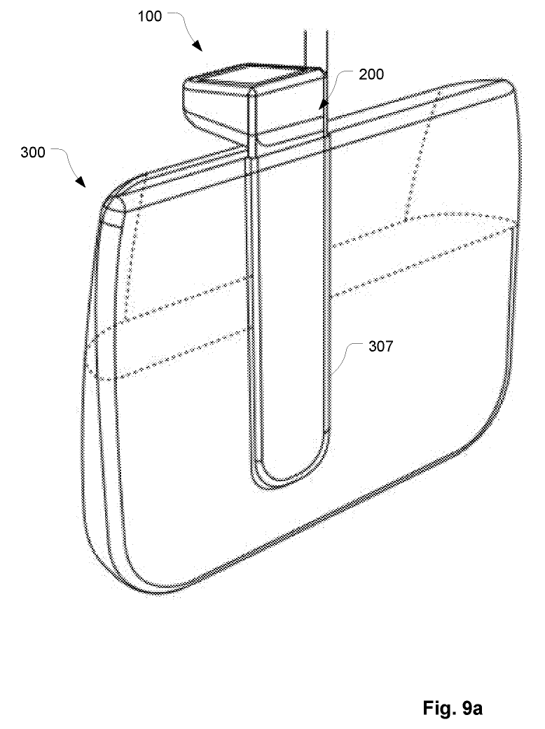

FIG. 8 shows a control device, a fill level measurement arrangement and a container according to an example.

The fill level measurement arrangement 200 and the control device 100, which are designed as shown in FIG. 7, are shown on the container 300. The container is designed as shown in FIG. 6a.

The fill level measurement arrangement 200 abuts the rear of the container 300. The connection between the container 300 and the fill level measurement arrangement 200 can be an adhesive connection.

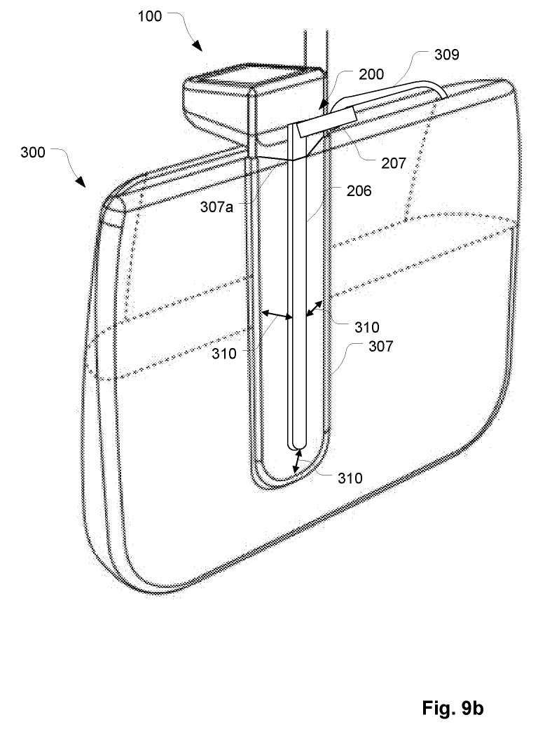

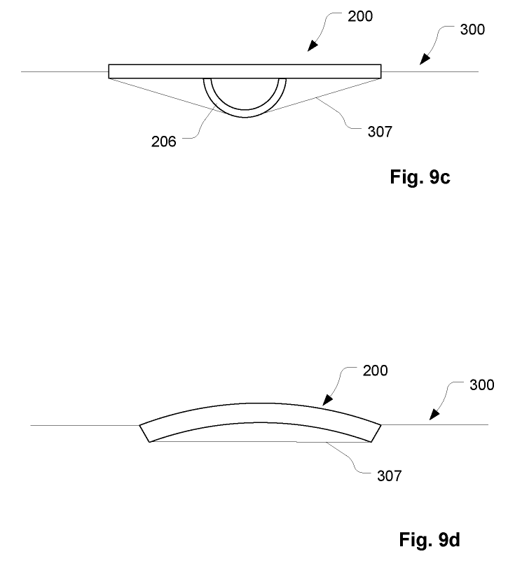

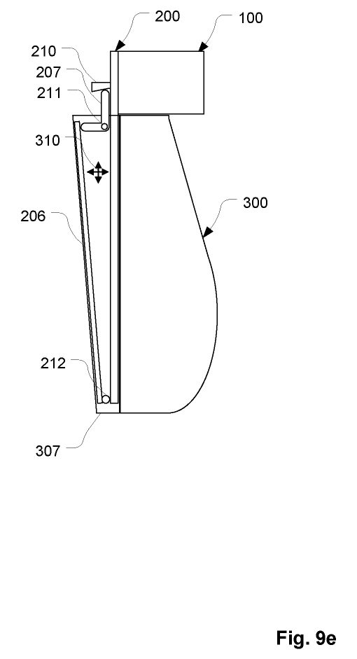

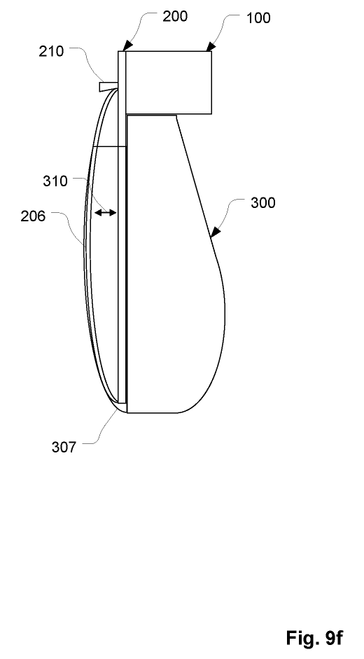

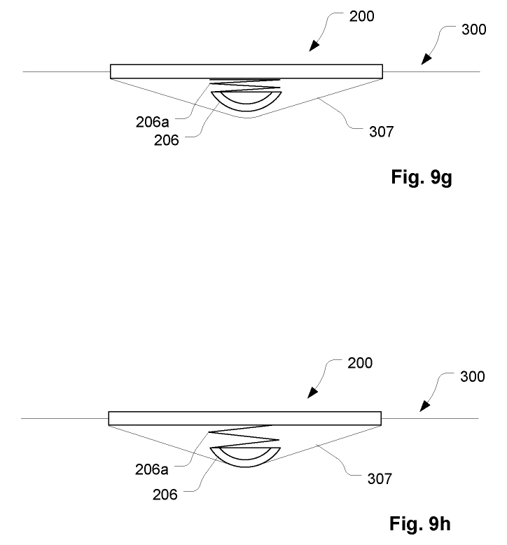

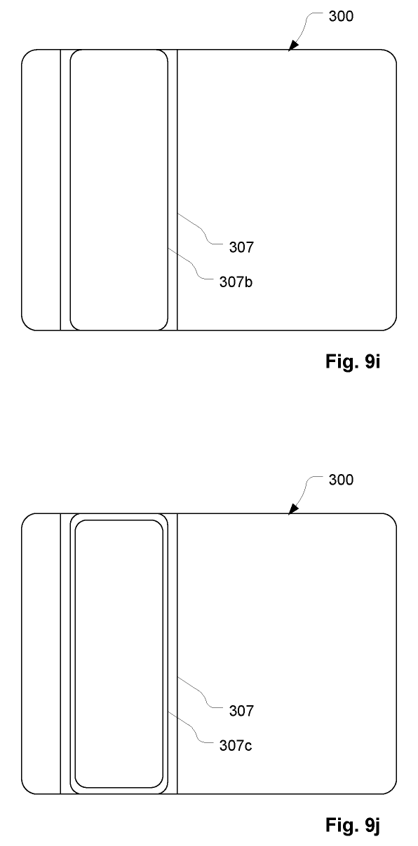

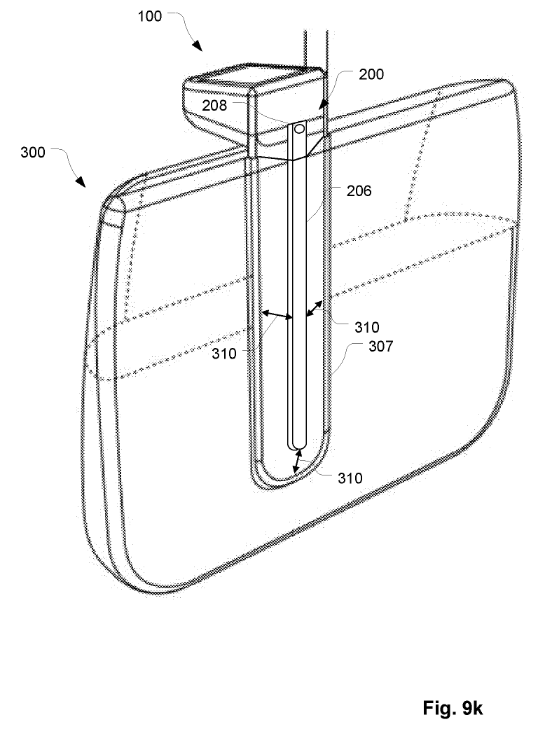

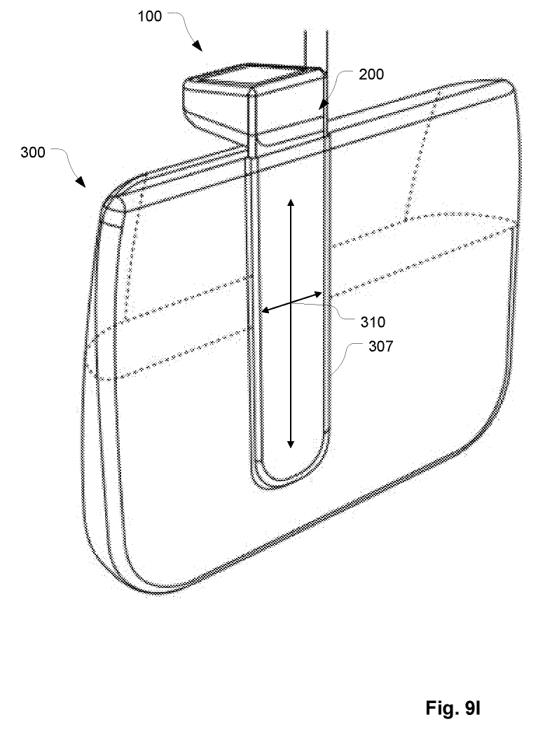

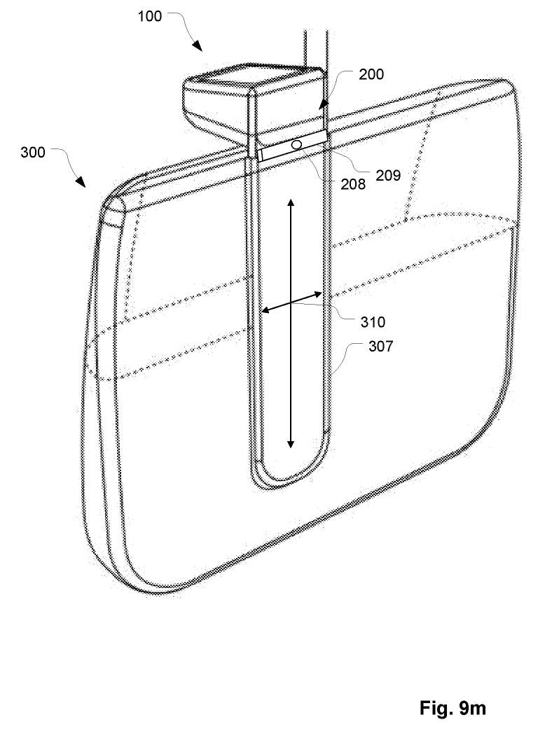

FIG. 9a shows a control device, a fill level measurement arrangement and a container according to an example.

The fill level measurement arrangement 200 and the control device 100, which are designed as shown in FIG. 7, are shown on the container 300. The container is designed as shown in FIG. 6a.

The fill level measurement arrangement 200 is contained in a flexible pouch 307 of the container 300. The fill level measurement arrangement 200 and the pouch 307 are connected according to the sword-sheath principle and thus the fill level measurement arrangement 200 abuts the rear of the container 300.

The pouch 307 may be formed of the same material as the outer wall of the container 300 and may be welded to the outer wall of the container.

FIG. 9b shows a control device, a fill level measurement arrangement and a container according to an example.

The control device 100 is designed as shown in FIG. 9a. The fill level measurement arrangement 200 is contained in the pouch 307 and, in addition to the example as shown in FIG. 9a, comprises a rear clamping element 206 and a latching element 207. In addition to the example shown in FIG. 9a, the container 300 comprises a counter bearing 309 designed as a carrying device.

The latching element 207 is connected to the rear clamping element 206. The latching element 207 latches into the counter bearing 309 of the container 300, thereby pressing the rear clamping element 206 into the pouch 307. The latching element can have a recess on its surface, where it latches onto the counter bearing 309 of the container 300, in order to ensure that it latches. The rear clamping element 206 is designed along the rear side of the fill level measurement arrangement 200. The rear clamping element 206 is connected to or abuts the fill level measurement device 200.

Due to the dimensioning of the rear clamping element 206, when the rear clamping element 206 is inserted into the pouch 307 together with the fill level measurement arrangement 200, a clamping force is generated in the foil of the pouch by stretching the foil over the rear clamping element 206. The clamping force, shown by the arrows 310, stretches the foil, which is disposed towards the exterior of the container. The horizontal clamping force can be generated solely by the geometric shape of the rear clamping element 206, in that the circumference of the pouch is smaller than the circumference required to wrap around the fill level measurement arrangement 200 and the rear clamping element 206. The clamping force is generated by the stretch in the material of the pouch. The latching element 207 can also be used to generate a vertical clamping force by pressing the fill level measurement arrangement 200 and the rear clamping element 206 into the pouches.

Furthermore, the clamping force also stretches the foil towards the interior of the container 300 over the surface of the fill level measurement arrangement 200 there. The clamping force prevents air bubbles from forming between the foil and the fill level measurement arrangement 200. Such air bubbles have the dielectric constant of air, which has an insulating effect in a capacitive measurement method of the fill level in the container 300 and therefore interferes with the measurement. Thus, the fill level measurement is improved.

In order to further improve the outflow of air from potential air bubbles between the foil and the surface of the fill level measurement arrangement 200, the surface can be provided with a structure featuring air channels. Furthermore, the fill level measurement arrangement 200 may also have holes that allow air to flow out of the air bubbles.

Because the air now collects in the pouch on the rear side of the fill level measurement arrangement 200, on which the rear clamping element 206 is formed, there is an insulating effect on the rear side due to the air having the corresponding dielectric constant. However, as this side faces the exterior of the container 300, the measurement should not be influenced by this area. Shielding this area via the air in the pouch is therefore a further improvement in the measurement. Thus, influences from a patient bed, for example, which may be located in this area, can be shielded.

FIG. 9c shows a cross-section through the pouch of the container with the fill level measurement arrangement and the rear clamping device according to the example shown in FIG. 9b.

A section of the wall of the container 300 is shown, wherein above the wall is the interior of the container 300 and below the wall is the exterior outside the container 300. The fill level measurement arrangement 200 and the rear clamping device 206 are disposed in the pouch 307. The foil is stretched over the rear clamping device 206 and thus abuts flat against the surface of the fill level measurement arrangement 200, wherein the surface against which the foil abuts faces the interior of the container 300.

Thus, as described with reference to FIG. 9b, air bubbles are displaced between the foil of the pouch 307 and the surface of the fill level measurement arrangement 200. Here, the foil of the pouch 307 is that which lies between the fill level measurement arrangement 200 and the inside of the container 300. Thus, an air cushion is also generated in the pouch 307 between the fill level measurement arrangement 200 and the outside of the container 300. Furthermore, the rear clamping device 206 has a cross-section similar to that of a half-tube, as a result of which the air cushion is also disposed within the rear clamping device 206. Thus, a disruption of the fill level measurement by air bubbles is avoided and the exterior is shielded.

FIG. 9d shows a cross-section through the pouch of the container with the fill level measurement arrangement according to an example.

Alternatively, a clamping force can also be generated by a curved fill level measurement arrangement 200. The fill level measurement arrangement 200 can also be flexible and elastic, thus generating the clamping force itself. By bending the fill level measurement arrangement 200, the foil is pulled smoothly over the surface of the fill level measurement arrangement 200 towards the inside of the container 300, causing the air bubbles between them to be displaced. Furthermore, the bend also forms an air cushion towards the outside of the container 300. Thus, a disruption of the fill level measurement by air bubbles is avoided and the exterior is shielded.

FIG. 9e shows a control device, a fill level measurement arrangement and a container according to an example in a side view.

The control device 100 is designed as shown in FIG. 9a. The fill level measurement arrangement 200 is contained in the pouch 307 and, in addition to the example as shown in FIG. 9a, comprises a rear clamping element 206, a latching element 207 and a latching device 210.

The clamping element 206 is rotatably mounted at the base (via a swivel joint 212) of the fill level measurement arrangement 200. The latching element 207 is designed as a rotatable lever (rotates around swivel joint 211) and can generate or vary the clamping force 310 by turning it over.

The latching element 207 can strut the rear clamping element 206 apart from the fill level measurement arrangement 200, so that the pouch 307 is stretched by the distance from the rear clamping element 206 from the fill level measurement arrangement 200, thereby generating the clamping force.

The latching element 207, which is designed as a lever, enables the fill level measurement arrangement 200 to be easily pushed into the pouch 307 without tension and the clamping force to be built up by turning over the latching element 207 in a pushed-in end position.

When the latching element 207 is turned over, it engages in an end position of the turning-over movement in the latching device 210. The latching device 210 can be designed to be on the fill level measurement arrangement 200, the control device 100 or the container.

If the latching device 210 is designed to be on the container 300, the latching element 207 is prevented from being turned over before reaching the pushed-in end position, since the latching element 207 can only be latched into the latching device 210 in this end position, whereby a vertical clamping force (310) is also built up.

Here, the latching device 210 can be designed as a form of counter bearing on the container 300, such as the counter bearing 309 designed as a carrying device as in FIG. 9b. In this case, the counter bearing generates the vertical clamping force 310.

FIG. 9f shows a control device, a fill level measurement arrangement and a container according to an example in a side view.

Compared to the example in FIG. 9e, the clamping element 206 is designed to be bendable and also fulfills the function of the latching element, whereby the latching element is comprised in the clamping element 206.

The clamping element 206 is disposed at the base of the fill level measurement arrangement 200 (can be mounted there via a swivel joint).

When the fill level measurement arrangement 200 is inserted into the pouch 307 together with the clamping element 206, the fill level measurement arrangement 200 and the clamping element 206 extend close to each other (substantially parallel), for example such that the pouch 307 is not stretched. Once the fill level measurement arrangement 200 has reached a pushed-in end position in the pouch 307, the bendable clamping element 206 can be pushed further into the pouch. In doing so, the clamping element 206 moves away from the fill level measurement arrangement 200, for example by forming an arc. Thus, in the bent state of the bendable clamping element 206, due to the distance of the bendable clamping element 206 from the fill level measurement arrangement 200, the pouch is stretched and a clamping force 310 is generated.

The upper free end of the clamping element 206 is then latched into the latching device 210. The latching device 210 can be designed to be on the fill level measurement arrangement 200, the control device 100 or the container.

If the latching device 210 is designed to be on the container 300, a vertical clamping force (310) is also built up. Here, the latching device 210 can be designed as a form of counter bearing on the container 300, such as the counter bearing 309 designed as a carrying device as in FIG. 9b. In this case, the counter bearing generates the vertical clamping force 310.

FIG. 9g shows a cross-section through the pouch of the container with the fill level measurement arrangement according to an example.

Alternatively, a clamping force can also be generated by a spring device 206a. The spring device 206a can be comprised in the rear clamping device 206. The rear clamping device 206 can be inserted into or removed from the pouch 307 in a state in which the spring device 206a is compressed and thus biased. This can be achieved by the reduced height of the rear clamping device 206 above the fill level measurement arrangement 200. FIG. 9e shows the compressed state. The pouch 307 does not abut the rear clamping device 206 and a clamping force is not generated in this state.

FIG. 9h shows a cross-section through the pouch of the container with the fill level measurement arrangement according to the example shown in FIG. 9g.

As soon as the target position of the rear clamping device 206 is reached during insertion, the spring device 206a can be decompressed, wherein the spring device 206a expands and presses the clamping device 206 against the inner wall of the pouch 307. FIG. 9h shows the decompressed state of the spring device 206a, in which the clamping force in the pouch 307 is generated by the spring device 206a.

The spring device 206a can be compressed by hand, for example by a user. For example, by pressing on the rear clamping device 206. In addition, a holding mechanism may be provided that holds the rear clamping device in a position in which the spring device 206a is compressed. This holding mechanism can have a separate actuating mechanism with which the user can release the holding mechanism and thus allow the rear clamping device 206 to rebound from the held compressed position. This actuating mechanism can be coupled to a latching element (such as 207 in FIG. 9b). As soon as the latching element latches into the carrying device (such as 309 in FIG. 9b) of the container 300, the holding mechanism is released and thus the rear clamping device 206 can rebound from the held compressed position.

To remove the rear clamping device 206, the spring device 206a and the fill level measurement arrangement 200, the clamping element 207 can be pressed against the fill level measurement arrangement 200, compressing the spring device 206a. The rear clamping device 206 and the spring device 206a can be held in this position by the holding mechanism, which makes it easier to remove the rear clamping device 206, the spring device 206a and the fill level measurement arrangement 200 from the pouch 307.

The mechanism described could also be driven by actuators (electric, magnetic, pneumatic, hydraulic, piezoelectric, etc.) instead of the spring device 206a, as the fill level measurement arrangement 200 has its own power supply.

FIG. 9i shows a rear side of the container according to an example.

The rear side of the container 300 is shown from the outside of the container 300. The container comprises a pouch 307 and a stiffening 307b in the pouch 307. The stiffening 307b is formed in the pouch on the foil towards the interior of the container 300. Thus, when the fill level measurement arrangement is inserted, the stiffening 307b is located between the fill level measurement arrangement and the interior of the container. The stiffening 307b may be applied to the foil of the container 300 or the foil of the container 300 may be designed to be thicker, at least in the pouch 307, to create the stiffening 307b.

The stiffening 307b prevents wrinkles that can create air bubbles between the foil towards the interior of the container 300 and fill level measurement arrangement. Such air bubbles have the dielectric constant of air, which has an insulating effect in a capacitive measurement method of the fill level in the container 300 and therefore interferes with the measurement. Thus, the fill level measurement is improved.

FIG. 9j shows a rear side of the container according to an example.

The rear side of the container 300 is shown from the outside of the container 300. The container comprises a pouch 307 and a stiffening 307c in the pouch 307. Compared to the stiffening 307b in FIG. 9i, the stiffening 307c is designed as a frame. The stiffening 307c can stretch the foil towards the interior of the container 300 and thus prevent air bubbles between a fill level measurement arrangement and the foil towards the interior of the container 300. The stiffening 307c can thereby surround a contact area of an electrode array of the fill level measurement arrangement and thus build up a clamping force which smoothes the foil of the rear wall of the bag in the interior in order to avoid wrinkles in which air bubbles can collect.

FIG. 9k shows a control device, a fill level measurement arrangement and a container according to an example.

The example according to FIG. 9g is a modification of the example according to FIG. 9b. Here, the rear clamping element 206 has a check valve and a connection 208 and can be inflated via these in order to generate the clamping force within the pouch 307. For inflation, it is possible to connect a pump or a syringe to the connection, for example. During inflation, the rear clamping device expands and thus causes the material of the pouch 307 to stretch, generating the clamping force. The check valve maintains the pressure inside the rear clamping device 206. The inflatable rear clamping device 206 can also be combined with a latching element as shown in FIG. 9b.

FIG. 9l shows a control device, a fill level measurement arrangement and a container according to an example.

The pouch 307 is made of an elastic material and is dimensioned such that a clamping force is generated when the fill level measurement arrangement 200 is inserted into the pouch 307, thereby stretching it.

FIG. 9m shows a control device, a fill level measurement arrangement and a container according to an example.

A sealing area 209 is provided on the fill level measurement arrangement 200, which seals the pouch so that air can be extracted from the pouch via the connection 208 and a check valve. For extracting the air, it is possible to connect a pump or a syringe to connection 208, for example.

For rear clamping devices which can be inserted into the pouch separately from the fill level measurement arrangement 200, it is also the case that the friction of the fill level measurement arrangement 200 and rear clamping device with the interior of the pouch 307 is eased by inserting them separately or, in the case of disassembly, pulling them out separately.

An analogous effect to the improved measurement described with reference to FIGS. 9b to m can also be achieved for a fill level measurement arrangement 200 printed as electrode arrays on the container 300, e.g. with electronic ink or as disposable sensors glued to the container. Gluing or ducking can prevent air bubbles, but at the expense of reusability of the fill level measurement arrangement 200, which is then disposed of with the container. Shielding of the exterior can be achieved, for example, by gluing an air-retaining strip, such as a foam strip or bubble wrap, to the outside of the fill level measurement arrangement 200 on the container 300.

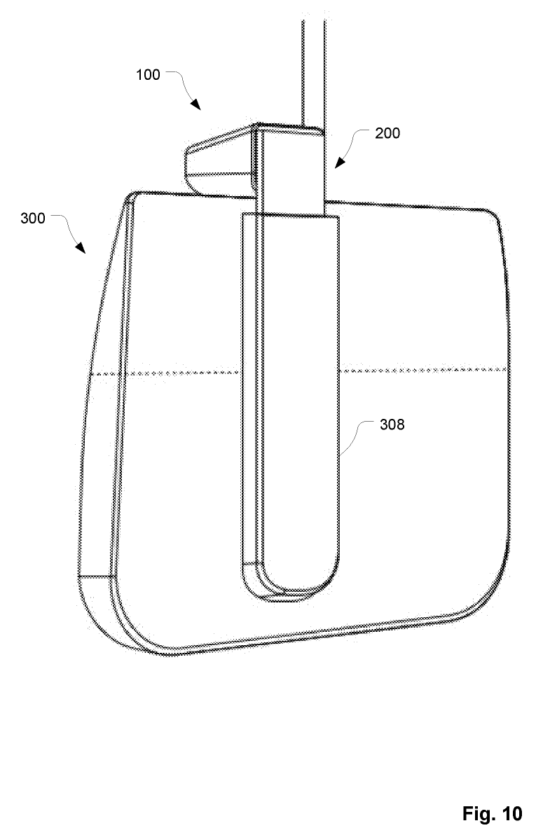

FIG. 10 shows a control device, a fill level measurement arrangement and a container according to an example.

The fill level measurement arrangement 200 and the control device 100, which are designed as shown in FIG. 7, are shown on the container 300. The container 300 is designed as shown in FIG. 6a.

The fill level measurement arrangement 200 is contained in a stiff pouch 308 of the container 300. The fill level measurement arrangement 200 and the pouch 308 are connected according to the sword-sheath principle and thus the fill level measurement arrangement 200 abuts the rear of the container 300.

The pouch 308 may be formed of a solid plastic and may be glued to the outer wall of the container, wherein the pouch provides stiffness to the container wall which improves the measurement accuracy of the fill level measurement due to a reduced curvature of the container wall.

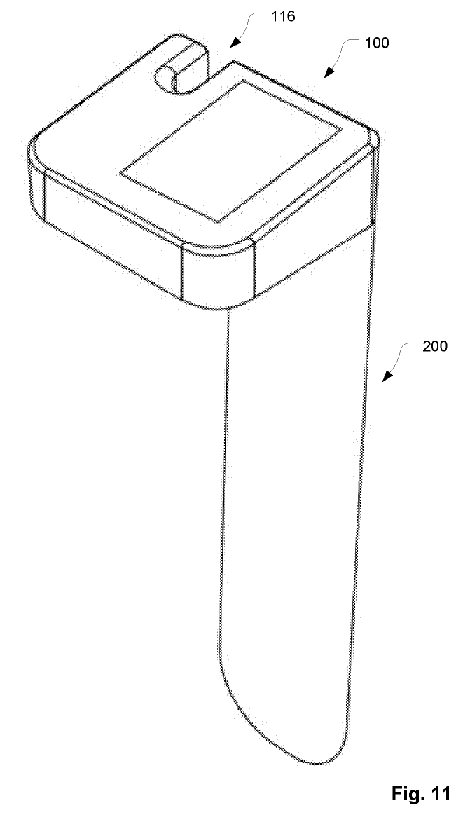

FIG. 11 shows a control device and a fill level measurement arrangement according to an example.

The fill level measurement arrangement 200 is connected to the control device 100 at the rear. This connection is designed, for example, using a fastening mechanism and a standardized interface. The fill level measurement arrangement 200 shown is designed as in FIG. 4a and the control device 100 shown is designed as in FIG. 3b with a diagnostic device 116.

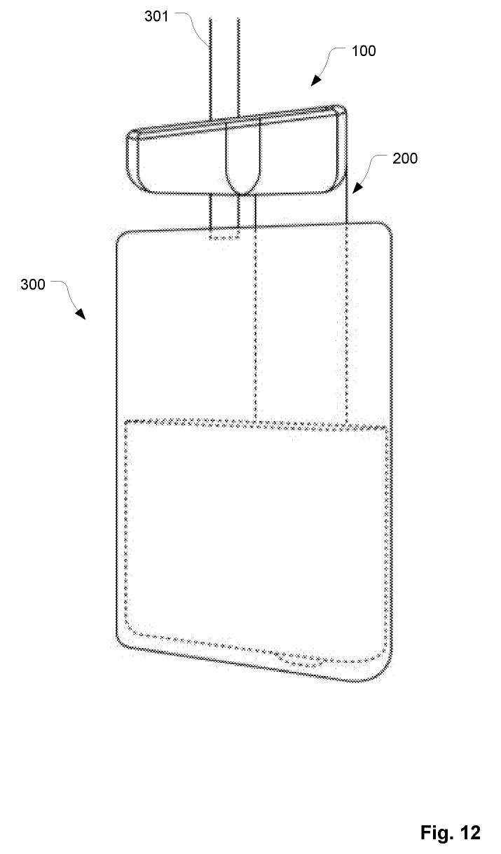

FIG. 12 shows a control device, a fill level measurement arrangement and a container according to an example.

The fill level measurement arrangement 200 and the control device 100, which are designed as shown in FIG. 11, are shown on the container 300. The container is designed as shown in FIG. 6b.

The fill level measurement arrangement 200 abuts the rear of the container 300. The connection between the container 300 and the fill level measurement arrangement 200 can be an adhesive connection.

The fill level measurement arrangement 200 and the container 300 can also be connected via a flexible pouch on the rear wall of the container, such as the connection already shown in FIG. 9.

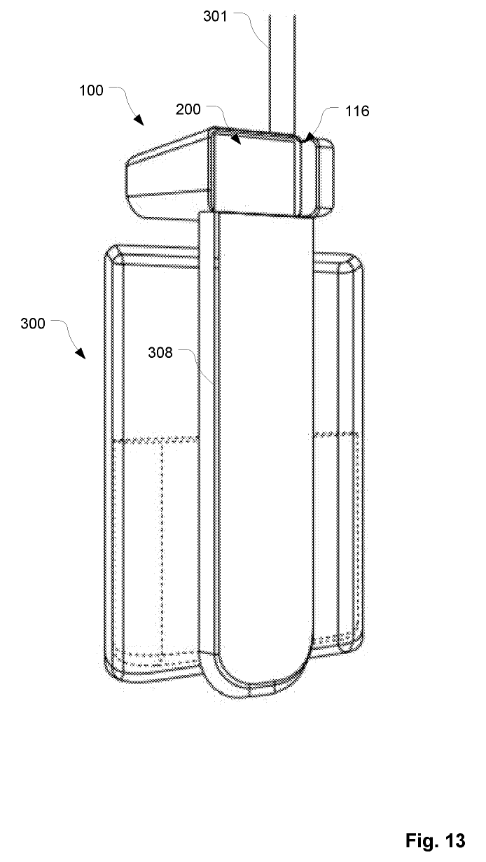

FIG. 13 shows a control device, a fill level measurement arrangement and a container according to an example.

The fill level measurement arrangement 200 and the control device 100, which are designed as shown in FIG. 11, are shown on the container 300. The container 300 is designed as shown in FIG. 6b.

The fill level measurement arrangement 200 is contained in a stiff pouch 308 of the container 300. The fill level measurement arrangement 200 and the pouch 308 are connected according to the sword-sheath principle and thus the fill level measurement arrangement 200 abuts the rear of the container 300.

The pouch 308 may be formed of a solid plastic and may be glued to the outer wall of the container, wherein the pouch provides stiffness to the container wall which improves the measurement accuracy of the fill level measurement due to a reduced curvature of the container wall.

The hose 301 of the container 300 is routed through the diagnostic device 116 in the position in which the container 300 is connected to the fill level measurement arrangement 200. Thus, fluid flowing through the hose 301 can be analyzed by the diagnostic device 116.

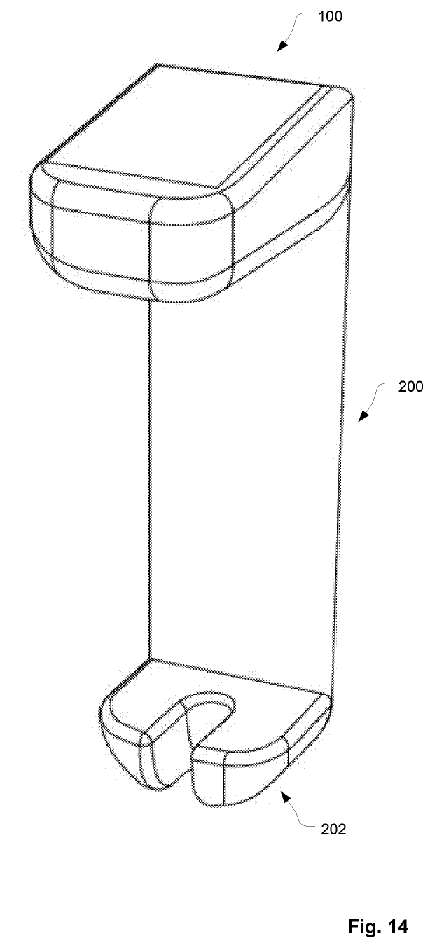

FIG. 14 shows a control device and a fill level measurement arrangement according to an example.

The fill level measurement arrangement 200 is connected to the control device 100 at the rear. This connection is designed, for example, using a fastening mechanism and a standardized interface. The fill level measurement arrangement 200 shown is designed as in FIG. 4b and the control device 100 shown is designed as in FIG. 3a. The fill level measurement arrangement 200 has a holder with actuator 202.

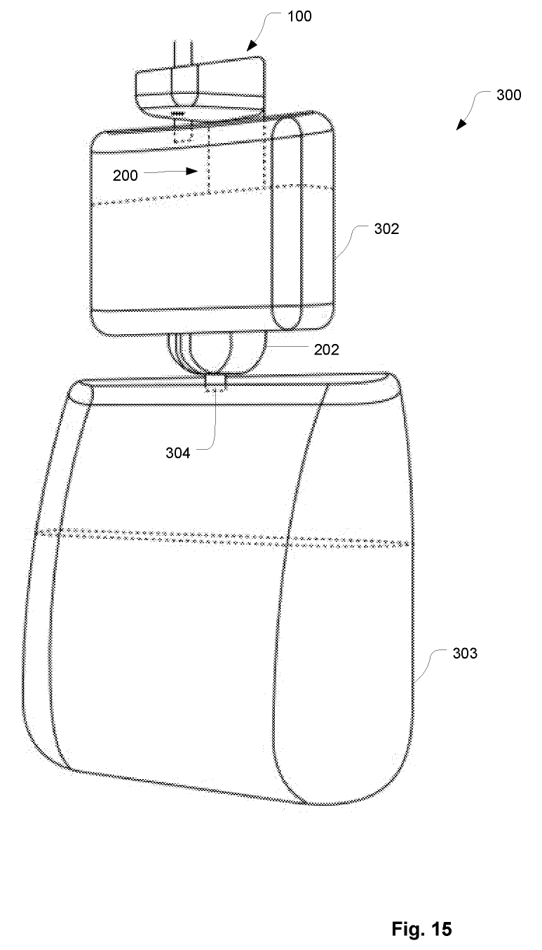

FIG. 15 shows a control device, a fill level measurement arrangement and a container according to an example.

The fill level measurement arrangement 200 and the control device 100, which are designed as shown in FIG. 14, are shown on the container 300. The container 300 is designed as shown in FIG. 6d.

The holder with actuator 202 of the fill level measurement arrangement 200 holds the container 300 at the transition 304 between the upper container 302 and the lower container 303. The fill level measurement arrangement 200 abuts the rear of the upper container 302 and measures the fill level there. In the upper container, the fill level of several columns can also be measured, as already described with reference to FIGS. 5d and 6d. The actuator 202 can close the transition 304 by pinching it.

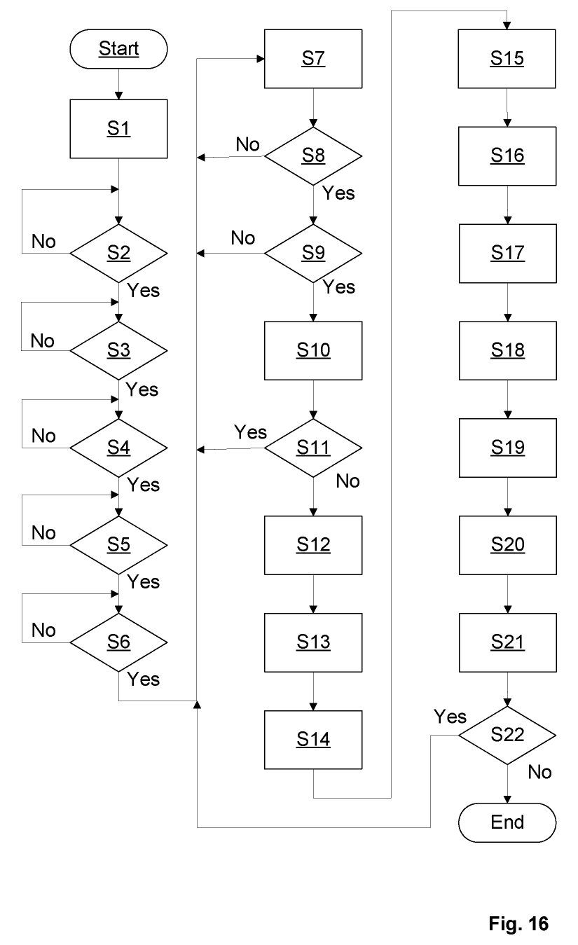

FIG. 16 shows a flow diagram of an exemplary fill level measurement.

The fill level measurement is carried out by the control device. After the start of the control device, the system is initialized in step S1. In step S2, it is checked whether a fill level measurement arrangement is connected and, if so, which one. If no fill level measurement arrangement is connected, step S2 is repeated and an error message may then be output. If a fill level measurement arrangement is connected, step S3 follows.

In step S3, it is checked whether the control device is connected to a network. If the control device is not connected to a network, step S3 is repeated and an error message may then be output. If the control device is connected to a network, step S4 follows.

In step S4, it is checked whether the control device is coupled to an electronic patient record (for example, on the server 401 in FIG. 1). If the control device is not coupled to an electronic patient record, step S4 is repeated and an error message may then be output. If the control device is coupled to an electronic patient record, step S5 follows.

In step S5, it is checked whether the container has been recognized (using its NFC tag). If the container is not recognized, step S5 is repeated and an error message may then be output. If the container is recognized, step S6 follows.

In step S6, it is checked whether the container is new, is an approved container, and is compatible with the fill level measurement arrangement. In addition, a mathematical conversion function is loaded that depends on the geometry of the container and the fill level measurement arrangement, which is required in steps S12 to S17 to convert and correct the measurement data. If this is not the case, step S6 is repeated and an error message may then be output. If this is the case, step S7 follows.

In step S7, the measurement begins. Here, capacitive measurement values of the fill level measurement arrangement are recorded, but these are not yet released. Furthermore, measurement values of an inertial measurement unit (IMU) are recorded.

In step S8, it is checked whether the position of the control device measured by the IMU and thus also of the container lies within a tolerance range around a reference position (vertical). If the position is not within the tolerance range, step S7 follows. When jumping to step 7, an error message may be output. If the measured position lies within the tolerance range, step S9 follows.

In step S9, it is checked whether the acceleration measured by the IMU lies within a tolerance range. If the acceleration does not lie within the tolerance range, step S7 follows. When jumping to step 7, an error message may be output. If the measured acceleration lies within the tolerance range, step S10 follows.

In step S10, the measurement values of the fill level measurement arrangement are recorded and analyzed as a plausibility measurement.

In step S11, the plausibility measurement is used to check whether an excessive change in the fill level has been measured compared to a previously measured fill level (comparison of the change against a threshold value). If an excessive change is detected, step S7 follows. When jumping to step 7, an error message may be output. If no excessive change is detected, a release criterion is issued for the measurement values of the fill level measurement arrangement in the plausibility measurement.

In step S12, the capacitive measurement values from the plausibility measurement are converted into a fill level. For this purpose, the conversion function loaded in S6 is used, which depends on the geometry of the detected containers and fill level measurement arrangement.

In step S13, the determined fill level is converted into a volume. For this purpose, the conversion function loaded in S6 is used, which depends on the geometry of the detected containers and fill level measurement arrangement.

In step S14, a deviation of the position measured by the IMU from the reference position is determined. For this purpose, the conversion function loaded in S6 is used, which depends on the geometry of the detected containers and fill level measurement arrangement.

In step S15, the determined volume is corrected based on the determined deviation. For this purpose, the conversion function loaded in S6 is used, which depends on the geometry of the detected containers and fill level measurement arrangement.

In step S16, the temperature of the liquid and the ambient temperature are recorded.

In step S17, a further correction of the corrected volume is performed using the temperature measurement values. Thus, a corrected fluid volume is obtained. For this purpose, the conversion function loaded in S6 is used, which depends on the geometry of the detected containers and fill level measurement arrangement.

In step S18, the corrected fluid volume is stored locally on the control device.

In step S19, the corrected fluid volume is displayed on the display.

In step S20, the corrected fluid volume is transmitted to the server.

In step S21, the corrected fluid volume is transmitted (saved) to the patient record.

In step S22, it is checked whether a further measurement should be performed. If a further measurement is to be performed, the procedure continues with step S7. If no further measurement is to be performed, the measurement ends.

FIG. 17 illustrates an example of a multi-purpose computer 500. The multi-purpose computer 500 is an example of a control device according to FIG. 2, which comprises a circuit that is configured to execute the method according to the present technology (e.g., the method from Fig. 17).

Examples that use software, firmware, programs, or the like to execute the methods described herein may be installed on the computer 500, which is then suitably configured for the corresponding example.

The computer 500 has a central processing unit (CPU) 501, which can execute various types of procedures and methods as described herein, for example according to programs which are stored in a read-only memory (ROM) 502; which are stored in a memory 507 and loaded into a random access memory (RAM) 503; which are stored on a medium 510 that can be inserted into a corresponding drive 509; etc.

The CPU 501, the ROM 502, and the RAM 503 are connected to a bus 511, which in turn is connected to an input/output interface 504. The number of CPUs, RAMs, and memories is only an example, and those skilled in the art will understand that the computer 500 can be adapted and configured accordingly to meet specific requirements that arise when it is used as a control device in accordance with the present technology.

Several components are connected to the input/output interface 504: an input 505, an output 506, a memory 507, a communication interface 508, and the drive 509, into which a medium 510 (compact disc (CD), digital video disc (DVD), CompactFlash memory, or the like) can be inserted.

The input 505 may comprise a pointing device (mouse, graphics tablet, or the like), a keyboard, a microphone, a camera, a touch screen, an eye-tracking unit, etc. Furthermore, the input can receive all measurement values from sensors described in FIG. 2.

The output 506 may include a display (liquid crystal display (LCD), cathode ray tube (CRT), light emitting diode (LED), etc.; e.g., included in a touch screen), loudspeaker, etc. Furthermore, the output can send control signals to actuators, as described in Fig. 2.

The memory 507 may comprise a hard disk drive, a solid-state drive (SSD), a flash drive, and the like.

The communication interface 508 may be adapted to communicate, for example, via a local area network (LAN), wireless local area network (WLAN), mobile telecommunications system (GSM, UMTS, LTE, NR, etc.), Bluetooth, near-field communication (NFC), infrared, etc.

It should be noted that the above description relates only to an example configuration of computer 500. Alternative configurations can be implemented with additional or different sensors, storage devices, interfaces, or the like. For example, the communication interface 158 may support wireless access technologies other than the aforementioned UMTS, LTE, and NR.

The invention being thus described, it will be obvious that the same may be varied in many ways. Such variations are not to be regarded as a departure from the spirit and scope of the invention, and all such modifications as would be obvious to one skilled in the art are to be included within the scope of the following claims.

What is claimed is:

1. A fill level determination system for determining a fill level criterion of a medical fluid in a container configured to hold a medical fluid, the fill level determination system comprising:
a fill level measurement arrangement configured to record measurement values corresponding to a fill level criterion of the container;
a detection device for determining a release criterion for a fill level determination system that determines a fill level of a medical fluid in the container;
a position detector configured to determine a position of the fill level determination system;
an acceleration detector configured to determine an acceleration of the fill level determination system, wherein fill level measurement values of the fill level determination system are recorded as a plausibility measurement, if the determined position lies within a tolerance range around a reference position and if the acceleration lies within a tolerance range, and the release criterion is granted on the basis of the plausibility measurement if a fill level measurement value of the fill level determination system does not show an excessive change from a previous fill level measurement value; and
a control device,
wherein the control device and the fill level measurement arrangement are configured to be connectable to each other and the fill level measurement arrangement and the container are configured to be connectable to each other,
wherein the determination of the fill level criterion takes place in a state of connection between the control device and the fill level measurement arrangement and between the fill level measurement arrangement and the container,
wherein the container comprises a holder, the fill level measurement arrangement and the container being configured to be connectable to each another via the holder, and
wherein the holder on the container comprises a flexible pouch that accommodates the fill level measurement arrangement based on a sword-sheath principle.

2. The fill level determination system according to claim 1, wherein the control device and the fill level measurement arrangement are configured to be connectable to each other via a standardized interface.

3. The fill level determination system according to claim 1, wherein fill level measurement arrangement comprises a rear clamping device that generates a clamping force in the flexible pouch.

4. The fill level determination system according to claim 3, wherein the container comprises a counter bearing that is configured to fasten the rear clamping device in the pouch.

5. The fill level determination system according to claim 4, wherein the rear clamping device is designed with a latching element, and wherein the latching element latches into the counter bearing so that the clamping force is generated.

6. The fill level determination system according to claim 1, wherein the container comprises a urine bag, a urine measurement system, an infusion bag, a thoracic drainage, a ventricular drainage, or a surgical drainage.

7. The fill level determination system according to claim 1, wherein the container comprises an RFID device, wherein the control device is configured to read the RFID device of the container via a further RFID device and to determine whether the container is compatible with the fill level measurement arrangement and the control device and has not yet been used, so that if the container is compatible and has not yet been used, it is written to the RFID device of the container via the RFID device that the container has been used and patient-related data is also written to the RFID device of the container.

8. The fill level determination system according to claim 1, wherein the container comprises an outlet valve, and wherein the fill level measurement arrangement comprises an actuator that is configured to actuate the outlet valve.

9. The fill level determination system according to claim 1, wherein the control device comprises a display that is configured to display information on the determined fill level criterion.

10. The fill level determination system according to claim 9, wherein the control device comprises a presence detector that is configured to detect an approach of a person or a gesture of a person, and wherein the display is switched on from a standby mode due to the approach or the gesture.

11. The fill level determination system according to claim 1, wherein the fill level measurement arrangement comprises electrodes that are configured to capacitively determine fill level measurement values.

12. The fill level determination system according to claim 1, wherein the fill level measurement arrangement comprises a printed circuit board and the electrodes are formed on the printed circuit board.

13. The fill level determination system according to claim 1, wherein the control device is configured to perform a correction of the fill level criterion on the basis of the determined position of the position detector of the detection device when the release criterion is granted.

14. A method for determining a release criterion for a fill level determination system according to claim 1, which determines a fill level of a medical fluid in a container, the method comprising:
determining a position of the fill level determination system;
determining an acceleration of the fill level determination system;
recording of fill level measurement values of the fill level determination system as a plausibility measurement if the determined position lies within a tolerance range around a reference position and if the acceleration lies within a tolerance range; and
granting the release criterion on the basis of the plausibility measurement if a fill level measurement value of the fill level determination system does not show an excessive change from a previous fill level measurement value.
